(19) United States
(12) Patent Application Publication
(10) Pub. No.: US 2010/0267606 A1
(43) Pub. Date: Oct. 21, 2010

(54) FRAGRANCING COMPOSITION COMPRISING AN AMPHIPHILIC COPOLYMER OF 2 ACRYLAMIDOMETHYLPROPANE-SULPHONIC ACID AND OPTIONALLY A CELLULOSE ALKYL ETHER AND/OR AN ALKYLCELLULOSE ALKYL ETHER

(75) Inventors: Frederique Tabarie, Savigny Sur Orge (FR); Elisabeth Picard-Lesboueyries, Velizy (FR)

Correspondence Address: CONNOLLY BOVE LODGE & HUTZ LLP, 1875 EYE STREET, N.W., SUITE 1100, WASHINGTON, DC, 20006, US

(73) Assignee: L'Oreal, Paris (FR)

(21) Appl. No.: 12/676,166

(22) PCT Filed: Sep. 8, 2008
(86) PCT No.: PCT/EP2008/061872

§ 371 Date: Jul. 1, 2010

Related U.S. Application Data
(60) Provisional application No. 60/996,206, filed on Nov. 6, 2007.
(60) Provisional application No. 60/996,208, filed on Nov. 6, 2007.

(30) Foreign Application Priority Data
Oct. 25, 2007 (FR) 0758577
Oct. 25, 2007 (FR) 0758579

(51) Int. Cl. A61K 8/81; A61Q 13/00

(52) U.S. Cl. 512/2

(57) ABSTRACT

The invention relates to a fragrancing composition comprising, in a cosmetically acceptable medium: a) at least 1% by weight relative to the total weight of the composition of at least one fragrancing substance; b) at least one volatile alcohol and/or one volatile silicone oil; c) at least one crosslinked or uncrosslinked amphiphilic copolymer of partially or completely neutralized 2-acrylamidomethylpropanesulphonic acid (AMPS) monomer (A) and at least one hydrophobic monomer (B) d) optionally at least a cellulose alkyl ether and/or an alkylcellulose alkyl ether, the alkyl radicals being linear or branched saturated C₁-C₄ alkyl radicals; and e) optionally water. The invention also relates to a method of fragrancing human keratinous substances and especially the skin, lips and integuments, comprising the application to the keratinous substances of the composition defined previously.

The invention relates to a fragrancing composition comprising, in a cosmetically acceptable medium:

a) at least 1% by weight relative to the total weight of the composition of at least one fragrancing substance;
b) at least one crosslinked or uncrosslinked amphiphilic copolymer of partially or completely neutralized 2-acrylamidomethylpropanesulphonic acid monomer (A) and at least one hydrophobic monomer (B)
d) optionally at least a cellulose alkyl ether and/or an alkylcellulose alkyl ether, the alkyl radicals being linear or branched saturated C₁-C₄ alkyl radicals; and
e) optionally water.

It is known that a fragrance is the combination of various odorous substances which evaporate at different times. Each fragrance exhibits what is termed a “top note” which is the odour that diffuses first when the fragrance is applied or on opening the receptacle containing it, a “heart or middle note” which corresponds to the complete fragrance (emitted for several hours after the “top note”) and a “base note” which is the most persistent odour (emitted for several hours after the “heart or middle note”). The persistence of the base note corresponds to the staying power of the fragrance.

Humans have always wanted to fragrance themselves and the objects surrounding them or the places around them, both in order to mask strong and/or unpleasant odours and to provide a pleasant odour.

Among the important quality criteria that are desired in perfuming products, are, on the one hand, the staying power of the fragrance, namely the persistence of the base note on the skin and, on the other hand, the transparency of the formulation and also its fluid nature for aesthetic reasons and reasons of ease of application.

The improvement of the staying power of fragrances has been the subject of many studies. It has been proposed in various patents and patent applications of the prior art to use various types of polymers, generally in perfuming hydroalcoholic solutions in order to improve the retention of the fragrance therein.

Thus, in U.S. Pat. No. 6,703,011, a system for the controlled release of fragrance by means of a permeable, continuous, non-porous, single-phase and emulsifier-free polymer film is proposed. The polymer film acts as a barrier for the fragrance which diffuses more slowly into the surrounding environment. Among the polymers used, polymers of the type LUVISKOL VA 55^{E}, ULTRAHOLD 8, LUVIQUAT FC or HM, ADVANTAGE PLUS, GAFQUAT 734N or 755N, DIAFORMERS, GAFQUAT HS-100, OMNIREZ 2000 are cited.

In Application WO 2004/098556, it is proposed to use, in a hydroalcoholic support, acrylic homopolymers or copolymers such as carbomers, pemulen or homopolymers or copolymers of the polyacryloyldimethyltaurate (AMPS) type such as HOSTACERIN AMP or ethylene oxide polymers in order to concentrate the fragrance molecules over a smaller surface area and extend the diffusion of said fragrance.

In Patent FR 2747306, it is proposed to use a non-ionic non-cyclic hydrocarbon-based polymer comprising anhydroglucose and/or ethylene units in a composition containing a fragrance for the fixing and/or prolonged release of this fragrance.

In Patent EP 804 924, it is proposed to use a polysaccharide alkyl ether comprising at least two different units and at least one hydroxyl group substituted by a saturated C₁-C₂₄ alkyl chain, in particular ethyl guar having a degree of ethyl substitution of at least 2, such as the commercial products AQUALON AQ-D3360-L and AQ-3360-H to increase the retention of a fragrance.

In U.S. Pat. No. 6,172,037, it is proposed to increase the staying power of a fragrance in a hydroalcoholic solution with a combination of a polyvinylpyrrolidone and a hydroxypropyl cellulose.

In Application WO 2005/070371, it is also proposed to use the combination of a carboxylated acrylates/-octacrylamide copolymer and of a hydrolysed jojoba ester in an alcoholic or hydroalcoholic composition for the purpose of reducing the fragrance concentration, of improving its retention on the skin and its resistance to water or sweat.

Although these polymers, as mentioned previously in the documents of the prior art, make it possible to increase the staying power of the fragrance, they generally have the drawback of substantially increasing the viscosity of hydroalcoholic lotions and/or of making them cloudy, opalescent and/or of forming precipitates in the solutions that make them not very attractive and unfit for consumption.

The need remains to find novel types of polymers that make it possible to effectively increase the staying power of the fragrance over a wide range of clear and fluid perfuming products such as eau de toilettes and eau de parfums without the drawbacks mentioned above, namely without adversely affecting the transparency, the stability, and the viscosity of the perfuming composition and without disturbing the well-being of the consumer such as the odour or the feel.

The Applicant has surprisingly discovered that this objective could be achieved using a partially or completely neutralized 2-acrylamidomethylpropanesulphonic acid amphiphilic polymer (AMPS) that is crosslinked or uncrosslinked with optionally a cellulose alkyl ether and/or an alkylcellulose alkyl ether

This discovery is the basis of the invention.

The invention relates to a fragrancing composition comprising, in a cosmetically acceptable medium:

a) at least 1% by weight relative to the total weight of the composition of at least one fragrancing substance;
b) at least one volatile alcohol and/or one volatile silicone oil;
c) at least one crosslinked or uncrosslinked amphiphilic copolymer of partially or completely neutralized 2-acrylamidomethylpropanesulphonic acid monomer (A) and at least one hydrophobic monomer (B);
d) optionally at least a cellulose alkyl ether and/or an alkylcellulose alkyl ether, the alkyl radicals being linear or branched saturated C₁-C₄ alkyl radicals; and
e) optionally water.

Another subject of the invention is a method of fragrancing human keratinous substances and especially the skin, lips and integuments (hair, eyebrows, eyelashes), comprising the application to the keratinous substances of the composition as defined above.

Another subject of the invention is the use of (a)) a crosslinked or uncrosslinked amphiphilic copolymer of partially or completely neutralized 2-acrylamidomethylpropanesulphonic acid monomer (A) and at least one hydrophobic monomer (B) and optionally b) a cellulose alkyl ether and/or an alkylcellulose alkyl ether, the alkyl radicals being linear or branched saturated C₁-C₄ alkyl radicals as defined previously in a fragrancing composition as defined above, for the purpose of increasing the staying power of the fragrance.

The expression “fragrancing substance” is understood to mean any fragrance or aroma capable of releasing a pleasant odour.

Preferably, the amount of fragrancing substance(s) varies from 1 to 30% by weight, better still from 2 to 25% by weight relative to the total weight of the composition.

The expression “cosmetically acceptable medium” is understood to mean, in the composition of the invention, a medium that is non-toxic and is capable of being applied to the skin (including inside the eyelids), the lips, nails or hair, eyelashes and eyebrows of humans.

The term “volatile alcohol” is understood to mean any compound comprising at least one alcohol functional group having a vapour pressure at 20° C. greater than 17.5 mmHg.

The term “oil” is understood, within the sense of the invention, to mean a fatty substance that is not soluble in water and that is liquid at ambient temperature and at atmospheric pressure.

The expression “volatile silicone oil” is understood, within the sense of the invention, to mean any silicone-based compound capable of evaporating in contact with the skin or with keratinous fibres in less than one hour, at ambient temperature and atmospheric pressure. The volatile compound is a volatile cosmetic compound that is liquid at ambient temperature and that especially has a non-zero vapour pressure at ambient temperature and atmospheric pressure, especially that has a vapour pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

Fragrancing substances that may be used in the composition of the invention include fragrances and aromas of natural or synthetic origin, and mixtures thereof. As fragrances and aromas of natural origin, mention may, for example, be made of extracts of flowers (lily, lavender, rose, jasmine, ylang-ylang), of stems and of leaves (patchouli, geranium, bitter leaf), of fruits (coriander, anise, cumin, juniper), of fruit peel (bergamot, lemon, orange), of roots (angelica, celery, cardamom, iris, sweet flag), of wood (pinewood, sandalwood, lignum vitae, pink cedar), of herbs and grasses (tarragon, lemongrass, sage, thyme), of needles and branches (spruce, fir, pine, dwarf pine), of resins and of balsams (galbanum, gum elemi, gum benzoin, myrrh, frankincense, opopanax).

As fragrancing substance of synthetic origin, mention may, for example, be made of compounds of ester, ether, aldehyde, ketone, aromatic alcohol and hydrocarbon type.

As esters, mention may in particular be made of benzyl acetate, benzyl benzoate, phenoxyethyl isobutyrate, p-tert-butylcyclohexyl acetate, citronellyl acetate, citronellyl formate, geranyl acetate, linalyl acetate, dimethylbenzylcarbinyl acetate, phenylethyl acetate, linalyl benzoate, benzyl formate, ethylmethylphenyl glycinate, alkylcyclohexyl propionate, styrallyl propionate and benzyl salicylate.

As ethers, mention may be made of benzyl ethyl ether.

As aldehydes, mention may, for example, be made of linear alkanals containing from 8 to 18 carbon atoms, citral, citronellal, cintronellyloxyacetaldehyde, cyclamenaldehyde, hydroxycitronellal, lilial and bourgeonal.

As ketones, mention may, for example, be made of ionones such as alpha-isomethylionone and methyl cedryl ketone.

As aromatic, and in particular terpenic, alcohols, mention may be made of anethol, citronellol, eugenol, isoeugenol, geraniol, linalol, phenylethyl alcohol and terpineol.

As hydrocarbons, mention may in particular be made of terpenes. These compounds are often in the form of a mixture of two or more of these odourous substances.

Moreover, it is also possible to use essential oils, and components of aromas, such as, for example, sage, camomile, clove, Melissa, mint, cinnamon leaf, lime blossom, juniper, vetiver, frankincense, galbanum, labolanum and lavandin essences.

As fragrancing substance, use is preferably made, alone or as a mixture, of essence of bergamot, dihydromyrcenol, lilial, lyral, citronellol, phenylethyl alcohol, alpha-hexylcinnamaldehyde, geraniol, benzylacetone, cyclamenaldehyde, linalol, ambroxan, indole, hedione, sandelice, lemon, mandarin and orange essences, allylamine glycolate, cyclovertal, essence of lavandin, essence of sage, beta-damascone, essence of geranium, cyclohexyl salicylate, phenylacetic acid, geranyl acetate, benzyl acetate and rose oxide.

According to a preferred embodiment of the invention, use is made of a mixture of various fragrancing substances which jointly generate a note that is pleasant to the user. Among the known olfactory notes, mention may, for example, be made of hesperide fragrances, aromatics, floral fragrances, musks, fruity fragrances, spicy fragrances, oriental fragrances, oceanic fragrances, aquatic notes, chypre fragrances, woody fragrances, fougere fragrances, and mixtures thereof.

### Amphiphilic AMPS Copolymers

The composition contains at least one crosslinked or uncrosslinked amphiphilic copolymer of partially or completely neutralized 2-acrylamidomethylpropanesulphonic acid monomer (A) and of at least one hydrophobic monomer (B), this copolymer being water-soluble or water-dispersible.

Furthermore, the expression “water-soluble or water-dispersible copolymer” is understood to mean a copolymer which, when introduced into water at a concentration equal to 1% by weight, results in a macroscopically homogeneous solution, the light transmittance of which, at a wavelength equal to 500 nm, through a 1 cm thick sample, is at least 10%, which corresponds to an absorbance [abs=−log (transmittance)] of less than 1.5.

The amphiphilic copolymers according to the invention generally have a number-average molecular weight ranging from 50 000 to 10 000 000, preferably from 100 000 to 8 000 000 and more preferably from 100 000 to 7 000 000.

The copolymers according to the invention are partially or completely neutralized by a mineral base (sodium hydroxide, potassium hydroxide, ammonium hydroxide) or an organic base such as monoethanolamine, diethanolamine or triethanolamine, an aminomethyl propanediol, N-methylglucamine, basic amino acids such as arginine and lysine, and mixtures thereof.

The amphiphilic copolymer used in the composition of the invention is capable of being obtained from:
partially or completely neutralized 2-acrylamidomethylpropanesulphonic acid monomer (A); and
one or more hydrophobic monomers (B) containing (i) an ethylenically unsaturated end group, (ii) a hydrophilic part of formula —(CH₂CH₂O)ₙ—(CH₂CH(CH₃)O)ₚ where n and p, independently of one another, denote an integer ranging from 0 to 30, preferably from 1 to 25 and more preferably from 3 to 20 on condition that n+p is less than or equal to 30, preferably less than and better still less than 20, which means that n+p may range from 0 to 30, preferably from 0 to 25 and better still from 0 to 20, and (iii) a hydrophobic part of CₘH₂ₘ₊₁ type that is aliphatic or cycloaliphatic, linear or branched, where m is an integer ranging from 6 to 22.

According to one particular embodiment of the invention, in the formula indicated above, the sum of n+p is an integer greater than or equal to 1 (n+p≧1).

The ethylenically unsaturated end group of the hydrophobic monomer B may especially be chosen from hydrophilic monomers having an ethylenic unsaturation, such as for example acrylic acid, methacrylic acid, acrylamide, methacrylamide, and mixtures thereof.

The hydrophobic part CₘH₂ₘ₊₁ comprises from 6 to 30 carbon atoms, preferably from 6 to 22 carbon atoms, more preferably from 6 to 18 carbon atoms and more preferably still from 12 to 18 carbon atoms. This hydrophobic part may be chosen, for example, from C₆-C₃₀, preferably C₆-C₁₈, alkyl radicals that are linear (for example, hexyl, octyl, decyl, dodecyl, hexadecyl, octadecyl, tetradecyl or oleyl), branched (for example, isostearic) or cyclic (for example, cyclododecane or adamantane). Among these hydrophobic radicals, linear and branched alkyl radicals are more particularly preferred.

The hydrophobic monomers (B) of the polymer used according to the invention are preferably chosen from acrylates of formula (I) below:

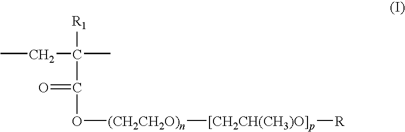

in which n and p, independently of one another, denote a number of moles that varies from 0 to 30, preferably from 1 to 25 and more preferably from 3 to 20, on condition that the sum n+p≧1 and less than or equal to 30, preferably less than 25 and better still less than 20; R₁ denotes a hydrogen atom or a linear or branched C₁-C₆ alkyl (preferably methyl) radical; and R denotes the CₘH₂ₘ₊₁ radical as defined above.

As indicated above, the amphiphilic copolymers may be crosslinked or uncrosslinked. According to one preferred embodiment of the invention, they are preferably uncrosslinked.

When the polymers are crosslinked, the crosslinking agents may be chosen from the compounds having an olefinic polyunsaturation commonly used for crosslinking polymers obtained by radical polymerization. Mention may be made, for example, as crosslinking agents, of divinylbenzene, diallyl ether, dipropylene glycol diallyl ether, polyglycol diallyl ethers, triethylene glycol divinyl ether, hydroquinone diallyl ether, tetraallyloxyethane, tetraethylene glycol diacrylate, triallylamine, trimethylolpropane diallyl ether, methylenebisacrylamide, allyl ethers of alcohols from the sugar series, allyl methacrylate, trimethylolpropane triacrylate (TMPTA) or mixtures thereof. The crosslinking agent is preferably chosen from methylenebisacrylamide, allyl methacrylate or trimethylolpropane triacrylate (TMPTA). The degree of crosslinking preferably varies from 0.01 to 10 mol % and more particularly from 0.2 to 2 mol % relative to the polymer.

The copolymers of the invention may additionally contain one or more other ethylenically unsaturated hydrophilic monomers, for example chosen from acrylic acid, methacrylic acid or their β-substituted alkyl derivatives or their esters obtained with monoalkylene glycols or polyalkylene glycols, acrylamide, methacrylamide, vinylpyrrolidone, itaconic acid, maleic acid and mixtures thereof.

The copolymers of the invention may moreover, additionally contain one or more ethylenically unsaturated hydrophobic comonomers, for example comprising:
a fluoro or C₆-C₁₈ alkylfluoro radical (for example the group of formula —(CH₂)₂—(CF₂)₉—CF₃);
a cholesteryl radical or a radical derived from cholesterol (for example, cholesteryl hexanoate);
a polycyclic aromatic group such as naphthalene or pyrene; and
a silicone or alkylsilicone or else alkylfluorosilicone radical.

According to one particular embodiment of the invention, the amphiphilic copolymers of AMPS used are uncrosslinked polymers, composed of

(A) 1 to 20 mol % and preferably 1 to 15 mol % of units of formula (I) as indicated above, and
(B) 80 to 99 mol % and preferably 85 to 99 mol % of 2-acrylamido-2-methylpropanesulphonic acid units of formula (II) below:

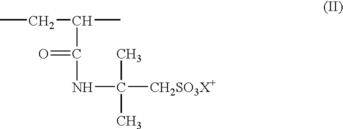

in which X⁺ is a proton, an alkali metal cation, an alkaline-earth metal cation or the ammonium ion.

The particularly preferred polymers are obtained from AMPS units of formula (II) and units of formula (I) where p=0; R₁ is the methyl (CH₃) radical; n is an integer ranging from 7 to 25 and R₂ is a C₁₀-C₁₈ alkyl radical.

The polymers for which X⁺ in the formula (II) denotes sodium or ammonium are more particularly preferred.

The amphiphilic polymers used according to the invention may be obtained according to conventional radical polymerization processes in the presence of one or more initiators such as, for example, azobisisobutyronitrile (AIBN), azobisdimethylvaleronitrile, 2,2-azobis[2-amidinopropane] hydrochloride (ABAH), organic peroxides such as dilauryl peroxide, benzyl peroxide, tert-butyl hydroperoxide, etc., mineral peroxide compounds such as potassium or ammonium persulphate, or H₂O₂ optionally in the presence of reducing agents.

The polymers may especially be obtained by radical polymerization in a tert-butanol medium in which they precipitate. By using polymerization via precipitation into tert-butanol, it is possible to obtain a distribution of the size of the polymer particles that is particularly favourable for its uses.

The polymerization reaction may be carried out at a temperature between 0 and 150° C., preferably between 10 and 100° C., either at atmospheric pressure, or under a reduced pressure. It may also be carried out under an inert atmosphere, preferably under nitrogen.

According to this process, the AMPS monomer of formula (II) with X⁺ denoting sodium or ammonium has especially been polymerized with an ester of acrylic acid or of methacrylic acid and
a C₁₀-C₁₈ alcohol oxyethylenated by 8 mol of ethylene oxide (GENAPOL® C-080 from HOECHST/CLARIANT);
a C₁₁ oxo alcohol oxyethylenated by 8 mol of ethylene oxide (GENAPOL® UD-080 from HOECHST/CLARIANT);
a C₁₁ oxo alcohol oxyethylenated by 7 mol of ethylene oxide (GENAPOL® UD-070 from HOECHST/CLARIANT);
a C₁₂-C₁₄ alcohol oxyethylenated by 3 mol of ethylene oxide (GENAPOL® LA-030 from HOECHST/CLARIANT);
a C₁₂-C₁₄ alcohol oxyethylenated by 7 mol of ethylene oxide (GENAPOL® LA-070 from HOECHST/CLARIANT);
a C₁₂-C₁₄ alcohol oxyethylenated by 9 mol of ethylene oxide (GENAPOL® LA-090 from HOECHST/CLARIANT);
a C₁₂-C₁₄ alcohol oxyethylenated by 11 mol of ethylene oxide (GENAPOL® LA-110 from HOECHST/CLARIANT);
a C₁₂-C₁₄ alcohol oxyethylenated by 25 mol of ethylene oxide (GENAPOL® LA-250 from HOECHST/CLARIANT);
a C₁₆-C₁₈ alcohol oxyethylenated by 8 mol of ethylene oxide (GENAPOL® T-080 from HOECHST/CLARIANT);
a C₁₆-C₁₈ alcohol oxyethylenated by 15 mol of ethylene oxide (GENAPOL® T-150 from HOECHST/CLARIANT);
a C₁₆-C₁₈ alcohol oxyethylenated by 11 mol of ethylene oxide (GENAPOL® T-110 from HOECHST/CLARIANT);
a C₁₆-C₁₈ alcohol oxyethylenated by 20 mol of ethylene oxide (GENAPOL® T-200 from HOECHST/CLARIANT);
a C₁₆-C₁₈ alcohol oxyethylenated by 25 mol of ethylene oxide (GENAPOL® T-250 from HOECHST/CLARIANT); and
a C₁₂-C₁₅ alcohol oxyethylenated by 23 mol of ethylene oxide.

According to one preferred embodiment of the invention, the amphiphilic polymer used is preferably an uncrosslinked copolymer of a monomer of formula (II) where X⁺ denotes sodium or ammonium and of a C₁₂-C₁₄ or C₁₂-C₁₅ alcohol methacrylate comprising 7 or 23 oxyethylenated groups of formula (I), obtained from methacrylic acid or from a salt of methacrylic acid and Genapol LA-070 or from a C₁₂-C₁₅ alcohol oxyethylenated by 23 mol of ethylene oxide.

More preferably, the amphiphilic polymers used are copolymers of monomer (II) with X⁺ denoting sodium or ammonium and of alcohol methacrylate having the following characteristics:

| | | | Molar proportion |
|---|---|---|---|
| | | | of monomer of |
| Name of pendent | | | formula |
| chains | R | n | (I) |
| Genapol LA-070 | C₁₂₋₁₄ linear alkyl | 7 | 8.5% |
| | chain | | |
| Genapol LA-070 | C₁₂₋₁₄ linear alkyl | 7 | 18.8% |
| | chain | | |
| C₁₂-C₁₅ alcohol | C₁₂₋₁₅ branched | 23 | 10.5% |
| oxyethylenated | alkyl chain | | |
| by 23 mol of | | | |
| ethylene oxide | | | |

More preferably still, use will be made of an amphiphilic copolymer of monomer (II) where X⁺ denotes sodium or ammonium and 18.8 mol % of alcohol methacrylate and of C₁₂-C₁₄ alcohol methacrylate comprising 7 oxyethylenated groups of formula (I) (Genapol LA-70).

The AMPS polymers according to the invention are generally present in amounts of active material ranging from 0.01 to 5% by weight, more preferably still from 0.1 to 5% by weight and more preferably still from 0.3 to 2% by weight relative to the total weight of the composition.

### Cellulose Compounds

Among the cellulose alkyl ethers that can be used according to the invention, mention may be made of:
cellulose methyl ether (INCI name: Methylcellulose) such as the product sold under the trade name BENECEL METHYLCELLULOSE by Hercules Incorporated or the product sold under the trade name METHOCEL A by Amerchol Corporation; and
cellulose ethyl ether (INCI name: Ethylcellulose) such as the product sold under the trade name AQUALON ETHYLCELLULOSE by Hercules Incorporated or the product sold under the trade name ETHOCEL by Dow Chemical.

Among the alkylcellulose alkyl ethers that can be used according to the invention, mention may be made of ethylcellulose methyl ether (INCI name: Methyl Ethyl Cellulose) such as the product sold under the trade name CELACOL CTP 991 by Courtauld Chemicals.

Use will more particularly be made of cellulose ethyl ether (INCI name: Ethylcellulose).

The cellulose compounds according to the invention are generally present in amounts of active material ranging from 0.01 to 4% by weight, more preferably still from 0.1 to 3% by weight and more preferably still from 0.3 to 2% by weight relative to the total weight of the composition.

According to one particular embodiment of the invention, use will be made of a mixture composed of at least a) one crosslinked or uncrosslinked amphiphilic copolymer of partially or completely neutralized 2-acrylamidomethylpropanesulphonic acid monomer (A) and at least one hydrophobic monomer (B) with b) at least one cellulose alkyl ether and/or an alkylcellulose alkyl ether so as to improve the clarity of the composition and/or reduce or even eliminate the low-temperature precipitation phenomena which may be caused by certain fragrances and/or improve the stability of the composition during storage. The mixture is used at concentrations ranging from 0.01 to 2.5% relative to the total weight of the composition.

The fragrancing compositions are generally transparent. Their transparency is measured by a turbidity ranging from 1 to 200 NTU and preferably from 1 to 50 NTU; turbidity measured at 24 hours using a HACH—model 2100 P portable turbidimeter.

The compositions according to the invention are generally lotions and preferably have a viscosity ranging from 0.5 to 60 mPa·s and more preferably from 1 to 60 mPa·s, more preferably still from 3 to 30 mPa·s; the viscosity being measured using a Rheomat TVe-05, at 25° C., speed of rotation 200 rpm, spindle No. 1, 10 min. These low viscosities make it possible to package the compositions of the invention using mechanical pressurization or propellant gas devices so as to be applied in the form of fine particles (vaporization).

The cosmetically acceptable medium in accordance with the present invention contains at least one volatile alcohol and/or one volatile silicone oil and optionally water. Preferably, the medium of the composition contains water in an amount preferably ranging from 0.01% to 50% and more preferably from 0.5% to 25% by weight relative to the total weight of the composition.

The volatile alcohols in accordance with the present invention are preferably chosen from C₁-C₅ lower monoalcohols, and can be chosen from methanol, ethanol, propanol, isopropanol, n-butanol, isobutanol and t-butanol, and more particularly ethanol.

The volatile alcohol(s) is(are) preferably present in amounts ranging from 40% to 80%, and more preferably in amounts ranging from 55% to 80% by weight, relative to the total weight of the composition.

As volatile silicone oils, mention may, for example, be made of linear or cyclic volatile silicone oils, in particular those having a viscosity ≦6 centistokes (6×10⁻⁶ m²/s) and having in particular from 2 to 10 silicon atoms, these silicones optionally comprising alkyl or alkoxy groups containing from 1 to 22 carbon atoms. As a volatile silicone oil that can be used in the invention, mention may in particular be made of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane and mixtures thereof.

The volatile silicone oil(s) is(are) preferably present at from 40% to 98.5%, preferably in concentrations ranging from 10% to 80% by weight, relative to the total weight of the composition.

The composition of the invention may also comprise any additive normally used in the fragrance field, chosen in particular from cosmetic or dermatological active agents, emollients or demulcents such as sweet almond oil, apricot kernel oil, moisturizers such as glycerol, calmatives such as α-bisabolol, allantoin, aloe vera; vitamins, essential fatty acids, insect repellants, propellants, fillers, pearlescent agents, flakes, dyestuffs that are soluble in the support of the composition, colour stabilizers for the fragrance and mixtures thereof. When they are present in the composition of the invention, these additives may be present in an amount ranging from 0.001 to 10% and better still from 0.01 to 5% by weight relative to the total weight of the composition.

The composition of the invention may also comprise dyestuffs that are soluble in the support of said composition.

As soluble dyes in accordance with the invention, mention may be made of water-soluble or hydrophilic dyes, such as:

caramel, Yellow 5, Acid Blue 9/Blue 1, Green 5, Green 3/Fast Green FCF 3, Orange 4, Red 4/Food Red 1, Yellow 6, Acid Red 33/Food Red 12, Red 40, cochineal carmine (CI 15850, CI 75470), Ext. Violet 2, Red 6-7, Ferric Ferrocyanide, Ultramarines, Acid Yellow 3/Yellow 10, Acid Blue 3, Yellow 10.

The soluble dye or dyes in accordance with the invention are preferably present in amounts ranging from 10⁻⁵ to 1% of the total weight of the composition, preferably from 10⁻⁴ to 0.1% of the total weight of the composition.

As colour stabilizers for fragrances, mention will be made of tris(tetramethylhydroxypiperidinol) citrate such as the product sold under the name TINOGUARD Q by Ciba-Geigy, sodium benzotriazolyl butylphenol sulphonate such as the product sold under the name TINOGUARD HS by Ciba-Geigy; benzotriazolyl dodecyl p-cresol such as the product sold under the name TINOGUARD TL by Ciba-Geigy, such as the product sold under the trade name CIBAFAST H LIQUID by Ciba-Geigy or bumetrizole such as the product sold under the name TINOGUARD AS by Ciba-Geigy.

According to one particular form of the invention, use will also be made of at least one antioxidant and/or at least one peptizer so as to improve the clarity of the composition and/or reduce or even eliminate the low-temperature precipitation phenomena which may be caused by certain fragrances and/or improve the stability of the composition during storage.

Among the antioxidants, mention may be made, for example, of BHA (tert-butyl-4-hydroxyanisole), BHT (2,6-di-tert-butyl-p-cresol), tocopherols such as vitamin E and its derivatives such as tocopheryl acetate. They are used at concentrations ranging from 0.01% to 1% relative to the total weight of the composition.

Among the peptizers that can be used according to the invention, use will more particularly be made of hydrogenated castor oil that is oxyethylenated with 60 mol of ethylene oxide: INCI name: PEG-60 HYDROGENATED CASTOR OIL such as the product sold under the trade name CREMAPHOR RH60 by BASF. They are used at concentrations ranging from 0.1% to twice the concentration of fragrance concentrate relative to the total weight of the composition.

According to one particular form of the invention, use will also be made of at least one cellulose alkyl ether and/or an alkylcellulose alkyl ether so as to improve the clarity of the composition and/or reduce or even eliminate the low-temperature precipitation phenomena which may be caused by certain fragrances and/or improve the stability of the composition during storage. They are used at concentrations ranging from 0.01 to 2.5% relative to the total weight of the composition.

Among the cellulose alkyl ethers that can be used according to the invention, mention may be made of:
cellulose methyl ether (INCI name: Methylcellulose) such as the product sold under the trade name BENECEL METHYLCELLULOSE by Hercules Incorporated or the product sold under the trade name METHOCEL A by Amerchol Corporation; and
cellulose ethyl ether (INCI name: Ethylcellulose) such as the product sold under the trade name AQUALON ETHYLCELLULOSE by Hercules Incorporated or the product sold under the trade name ETHOCEL by Dow Chemical.

Among the alkylcellulose alkyl ethers that can be used according to the invention, mention may be made of ethylcellulose methyl ether (INCI name: Methyl Ethyl Cellulose) such as the product sold under the trade name CELACOL CTP 991 by Courtauld Chemicals.

Use will more particularly be made of cellulose ethyl ether (INCI name: Ethylcellulose).

Of course, a person skilled in the art will be sure to choose the optional additional additives and/or their amount in such a way that the advantageous properties of the composition according to the invention are not, or not substantially, impaired by the envisaged addition.

The invention applies not only to the coloured fragrancing products but also to care products, and products for treating the skin, including the scalp, and the lips, that contain an odorous substance. The composition according to the invention may thus constitute a fragrancing, care or treatment composition for keratinous substances, and especially may be in the form of eau fraîche (splash), eau de toilette, eau de parfum, aftershave lotion, skincare water, silicone or hydrosilicone care oil. It may also be in the form of a fragranced two-phase lotion (eau de toilette phase/hydrocarbon-based oil and/or silicone oil phase).

Another subject of the invention is a cosmetic method of fragrancing human keratinous substances and especially the skin, lips and integuments, comprising the application to the keratinous substances of the composition as defined above.

The composition according to the invention may be manufactured by known processes, generally used in the field of fragranced and coloured lotions.

The compositions according to the invention in the form of lotions may be packaged in the form of bottles. They may also be applied in the form of fine particles by means of mechanical pressurization or propellant gas devices. The devices according to the invention are well known to a person skilled in the art and comprise pump bottles or sprays, aerosol containers comprising a propellant and also aerosol pumps that use compressed air as a propellant. The latter are described in U.S. Pat. No. 4,077,441 and U.S. Pat. No. 4,850,517 (that are an integral part of the contents of the description).

The compositions packaged in an aerosol in accordance with the invention generally contain conventional propellants such as, for example, hydrofluoro compounds, dichlorodifluoromethane, difluoroethane, dimethyl ether, isobutane, n-butane, propane and trichlorofluoromethane.

The invention will now be described with reference to the following examples given by way of illustration and non-limitingly. In these examples, unless indicated otherwise, the amounts are expressed as weight percentages. The following fragrancing formulations were produced; the amounts are indicated in weight percentages:

### EXAMPLES

### Example 1

### Eau De Toilette

| | |
|---|---|
| Copolymer of monomer of formula (II) | 1.0% |
| and of GENAPOL LA-070 methacrylate | |
| (with 18.8 mol % of monomer of formula II) | |
| Fragrance Ô de Lancôme | 5.8% |
| 96° ethanol | 75.5% |
| BHT | 0.05% |
| Water | qs for 100% |

An eau de toilette was obtained that had a good retention (see olfactory tests below), was clear, stable during storage at ambient temperature, 37° C. and 45° C. However, a slight low-temperature flocculation phenomenon was observed at 4° C. but it was reversible from 12° C. A turbidity of 1 was obtained, measured at 24 hours using a portable Hach-model 2100 P turbidimeter. The viscosity measured using the Rheomat TVe-05, at 25° C., with a speed of rotation of 200 rpm, spindle No. 1, for 10 min was about 7 mPa·s DU.

### Example 3

### Eau De Toilette

| | |
|---|---|
| Copolymer of monomer of formula (II) | 0.5% |
| and of GENAPOL LA-070 methacrylate | |
| (with 18.8 mol % of monomer of formula I) | |
| Ethylcellulose | 0.5% |
| Fragrance Ô de Lancôme | 5.8% |
| 96° ethanol | 75.5% |
| BHT | 0.05% |
| Water | qs for 100% |

An eau de toilette was obtained that had a good retention (see olfactory tests below), was clear, stable during storage at low temperature (4° C.) and at ambient temperature, 37° C. and 45° C. No low-temperature flocculation phenomenon was observed at 4° C. A turbidity of 7 was obtained, measured at 24 hours using a portable Hach-model 2100 P turbidimeter. The viscosity measured using the Rheomat TVe-05, at 25° C., with a speed of rotation of 200 rpm, spindle No. 1, for 10 min was about 8 mPa·s.

### Example A Control

### Eau De Toilette

| | |
|---|---|
| Fragrance Ô de Lancôme | 5.8% |
| 96° ethanol | 75.5% |
| BHT | 0.05% |
| Water | qs for 100% |

### Sensory Tests Carried Out on the Eau De Toilettes Examples No. 1 and 2

The sensory tests were carried out using a panel of 7 to 9 experts trained to smell and evaluate the eau de toilettes. The olfactory evaluations were carried out on smelling strips at T=0, 1 hour, 4 hours, 6 hours and 24 hours.

For each composition 1 or 2, a triangular test was carried out with olfaction of 3 samples: 1 control and 2 test samples with composition 1 or 2 according to the invention.

The test was repeated 3 times at each time of analysis, the expert judge therefore dipped 9 smelling strips.

The percentage of people that recognize composition 1 or 2 of the invention as being more intense compared to the control sample was evaluated.

Example 1: panel of 7 judges
Example 2: panel of 9 judges

| | % of judges having assessed the composition | | | | |
|---|---|---|---|---|---|
| | of the invention as more intense compared | | | | |
| | to the control | | | | |
| Composition | T = 0 | 1 hour | 4 hours | 6 hours | 24 hours |
| Example 1 | 10% | 57% | 62% | 90% | 76% |
| Example 2 | 50% | 77% | 66% | 80% | 100% |

### Example 3

### Eau De Toilette

| | |
|---|---|
| Copolymer of monomer of formula (II) | 1.0% |
| and of GENAPOL LA-070 methacrylate | |
| (with 18.8 mol % of monomer of formula II) | |
| Fragrance Eau Vitale | 2% |
| 96° ethanol | 75.5% |
| PEG-60 HYDROGENATED CASTOR OIL | 0.5% |
| (CREMAPHOR RH60 by BASF) | |
| BHT | 0.05% |
| Water | qs for 100% |

An eau de toilette was obtained that had a good retention, was clear, stable during storage at ambient temperature, 37° C. and 45° C. Compared to Example 1, a substantial reduction in the low-temperature flocculation phenomenon at 4° C. was observed. A turbidity of 1 and a viscosity of about 7 mPa·s were obtained.

1. Fragrancing composition comprising, in a cosmetically acceptable medium: a) at least 1% by weight relative to the total weight of the composition of at least one fragrancing substance; b) at least one volatile alcohol and/or one volatile silicone oil; c) at least (i) one crosslinked or uncrosslinked amphiphilic copolymer of partially or completely neutralized 2-acrylamidomethylpropanesulphonic acid (AMPS) monomer (A) and at least one hydrophobic monomer (B) d) optionally at least a cellulose alkyl ether and/or an alkylcellulose alkyl ether, the alkyl radicals being linear or branched saturated C₁-C₄ alkyl radicals; and e) optionally water.

2. Composition according to claim 1, where the amount of fragrancing substance varies from 1 to 30% by weight relative to the total weight of the composition.

3. Composition according to claim 1, where the amphiphilic AMPS copolymer is present in amounts of active material ranging from 0.01 to 5% by weight relative to the total weight of the composition.

4. Composition according to claim 1, characterized in that the amphiphilic AMPS copolymer is composed of:
(A) 1 to 20 mol % of units of formula (I) below: 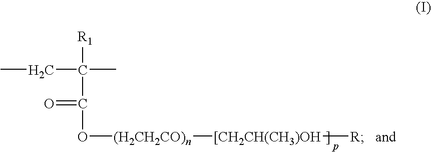
(B) 80 to 99 mol % of 2-acrylamido-2-methylpropanesulphonic acid units of formula (II) below: 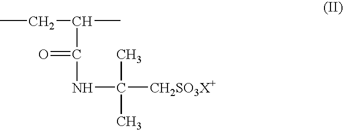
in which X⁺ is a proton, an alkali metal cation, an alkaline-earth metal cation or the ammonium ion.

5. Composition according to claim 4, where the amphiphilic AMPS copolymer is obtained from AMPS units of formula (II) and units of formula (I) where p=0; R₁ is the methyl (CH₃) radical; n is an integer ranging from 7 to 25 and R₂ is a C₁₀-C₁₈ alkyl radical.

6. Composition according to claim 4, where the amphiphilic copolymer is an uncrosslinked copolymer of a monomer of formula (II) where X⁺ denotes sodium or ammonium and of a C₁₂-C₁₄ or C₁₂-C₁₅ alcohol methacrylate comprising 7 or 23 oxyethylenated groups of formula (I).

7. Composition according to claim 1, where the volatile alcohol is chosen from C₁-C₅ lower monoalcohols.

8. Composition according to claim 7, where the volatile alcohol is ethanol.

9. Composition according to claim 1, where the cellulose alkyl ether and/or alkylcelullose alkyl ether are present in concentrations ranging from 0.1% to 2.5% relative to the total weight of the composition.

10. Composition according to claim 1, where the cellulose alkyl ether is chosen from cellulose methyl ether or cellulose ethyl ether.

11. Composition according to claim 1 where the alkylcellulose alkyl ether is ethylcellulose methyl ether.

12. Composition according to claim 1, comprising a mixture composed of at least a) one amphiphilic AMPS copolymer and of b) at least one cellulose alkyl ether and/or an alkylcellulose alkyl ether, the alkyl radicals being linear or branched saturated C₁-C₄ alkyl radicals.

13. Composition according to claim 12, where said mixture is present in concentrations ranging from 0.01 to 2.5% relative to the total weight of the composition.

14. A fragrancing composition as defined in claim 1 wherein at least an amphiphilic AMPS copolymer and/or at least one cellulose alkyl ether and optionally at least an alkylcellulose alkyl ether, the alkyl radicals being linear or branched saturated C₁-C₄ alkyl radicals increase the staying power of the fragrance.

15. Method of fragrancing human keratinous substances and especially the skin, lips and integuments, comprising the application to the keratinous substances of the composition as defined in claim 1.

16. Composition according to claim 1, where the amount of fragrancing substance varies from 2 to 25% by weight relative to the total weight of the composition.

17. Composition according to claim 1, where the amphiphilic AMPS copolymer is present in amounts of active material ranging from 0.1 to 5% by weight relative to the total weight of the composition.

18. Composition according to claim 1, where the amphiphilic AMPS copolymer is present in amounts of active material ranging from 0.3 to 2% by weight relative to the total weight of the composition.

19. Composition according to claim 2, where the amphiphilic AMPS copolymer is present in amounts of active material ranging from 0.01 to 5% by weight relative to the total weight of the composition.

20. Composition according to claim 2, where the amphiphilic AMPS copolymer is present in amounts of active material ranging from 0.1 to 5% by weight relative to the total weight of the composition.
